(19) United States
(12) Patent Application Publication
(10) Pub. No.: US 2013/0331813 A1
(43) Pub. Date: Dec. 12, 2013

(54) SYSTEM AND METHOD OF TRANS-ABDOMINAL PRE-AORTIC GANGLION ABLATION

(71) Applicant: ENIGMA MEDICAL, INC., San Diego, CA (US)

(72) Inventors: Denise Barbut, New York, NY (US); Allan Rozenberg, San Diego, CA (US); Axel Heinemann, New York, NY (US)

(73) Assignee: ENIGMA MEDICAL, INC., San Diego, CA (US)

(21) Appl. No.: 13/827,758

(22) Filed: Mar. 14, 2013

Related U.S. Application Data
(63) Continuation-in-part of application No. 13/787,325, filed on Mar. 6, 2013.
Application No. 13/787,325 is a continuation-in-part of application No. 13/787,358, filed on Mar. 6, 2013.
Application No. 13/787,358 is a continuation-in-part of application No. 13/787,375, filed on Mar. 6, 2013.
(60) Provisional application No. 61/641,599, filed on May 2, 2012.
(60) Provisional application No. 61/724,086, filed on Nov. 8, 2012.
(60) Provisional application No. 61/733,034, filed on Dec. 4, 2012.
(60) Provisional application No. 61/739,396, filed on Dec. 19, 2012.

(51) Int. Cl. A61B 18/18; A61M 37/00; A61B 17/32; A61B 18/14; A61B 18/20

(52) U.S. Cl. CPC A61B 18/18; A61B 18/14; A61B 18/20; A61B 17/320068; A61M 37/00
USPC 604/500; 606/41; 606/13; 606/33; 606/170

(57) ABSTRACT

A method of modulating a physiological parameter of a patient by percutaneously or transcutaneously disabling one or more pre-aortic ganglion cells within a pre-aortic ganglion via the anterior abdominal wall and improving the physiological parameter is provided. The pre-aortic ganglion cells may be disabled by applying radiofrequency, high intensity or low intensity focused ultrasound.

### RELATED APPLICATION DATA

This application claims priority to U.S. application Ser. No. 61/641,599, filed on May 2, 2012; and U.S. application Ser. No. 61/724,086, filed on Nov. 8, 2012; and U.S. application Ser. No. 61/733,034, filed on Dec. 4, 2012; and U.S. application Ser. No. 61/739,396, filed on Dec. 19, 2012; and is a continuation-in-part of U.S. application Ser. No. 13/787,325, filed on Mar. 6, 2013; and is a continuation-in-part of U.S. application Ser. No. 13/787,358, filed on Mar. 6, 2013; and is a continuation-in-part of U.S. application Ser. No. 13/787,375, filed on Mar. 6, 2013; the entireties of which are incorporated herein by reference.

### FIELD OF THE INVENTION

The present invention relates generally to the field of hypertension. More specifically, the present invention relates to a system and method of non-invasive pre-aortic ganglion ablation for the treatment of hypertension.

### BACKGROUND OF THE INVENTION

Hypertension affects tens of millions of individuals. Untreated hypertension is associated with stroke, heart failure and renal failure. Most patients with hypertension are currently treated pharmacologically, many with multiple medications. A quarter of these patients are resistant to medication and their blood pressure poorly controlled, putting them at added risk for complications.

Activation of the sympathetic nervous system is thought to play a significant role in exacerbating hypertension in the later stages of the disease. Reducing such sympathetic activation has been shown to reduce blood pressure in these circumstances.

Recently, mechanical ablation of the renal nerves surrounding the renal artery has been shown to reduce blood pressure in patients with resistant hypertension. The technique consists of an endovascular, arterial procedure and involves radiofrequency ablation of renal nerve fibres, accessed through the wall of the renal arteries bilaterally. Renal artery denervation, as the procedure is known, has been shown to reduce systolic and diastolic pressures by up to 30 mm and10 mm respectively, and to be persistent out to a year or more following the procedure. The incidence and severity of procedure related and late complications are as yet unknown, as is the long term benefit on blood pressure reduction since renal nerve fibres regenerate, and the hypotensive effect of this ablative procedure may diminish over time.

Therefore, alternatives to these therapies are needed which provide more significant reductions in blood pressure, persist indefinitely and which are safer, simpler, and less time-consuming.

### BRIEF SUMMARY OF THE INVENTION

The system and method of non-invasive or minimally invasive pre-aortic ganglion ablation offers a new effective method of controlling blood pressure in patients with medication resistant hypertension. It also overcomes the shortcomings of renal artery denervation. The present invention provides a system and method for ablating cell bodies within the pre-aortic ganglia transcutaneously or percutaneously for the treatment of hypertension and related disorders. These ganglionic cells can easily be accessed through the anterior abdominal wall. This method of treating hypertension has not been previously described.

### BRIEF DESCRIPTION OF THE DRAWINGS

For a better understanding of the invention, and to show how the same may be carried into effect, reference will now be made, by way of example, to the accompanying drawing:

FIG. 1 depicts a pre-aortic ganglion cell into which a radiofrequency probe is inserted percutaneously through the abdominal wall and radiofrequency energy transmitted to disable the pre-aortic ganglion cell.

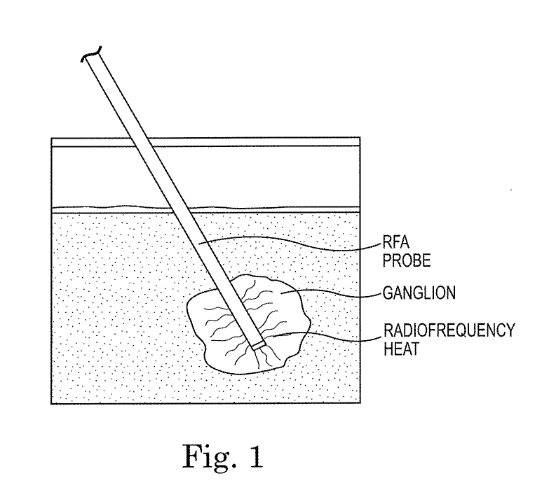

FIG. 2 depicts a pre-aortic ganglion cell into which high intensity focused ultrasound is being applied transcutaneously to disable the pre-aortic ganglion cell with damage to intervening tissue avoided.

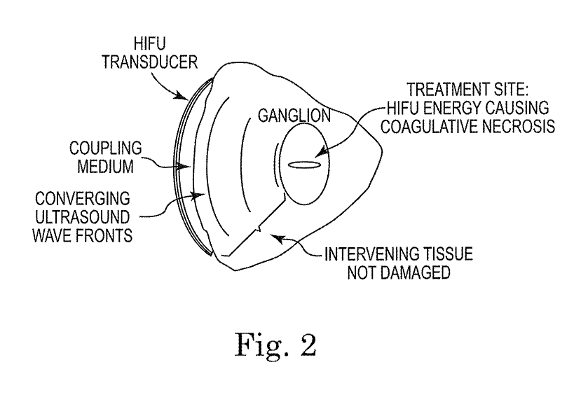

### DETAILED DESCRIPTION OF THE INVENTION

The present invention covers a system and method of denervating a portion of the cell bodies within the pre-aortic ganglia for the treatment of hypertension and related diseases. These ganglia can be accessed through the anterior abdominal wall. This method of treating hypertension in accordance with the invention has not been previously described.

Hypertension is one of the most common chronic conditions in the world. It affects one in every 7 people globally, or 1 billion people. In the US alone, it affects 1 in 4 adults, close to 70M people. In Europe and Japan, the prevalence is almost double that in the US, affecting 50% or more of adults. It is a major risk factor for heart disease, congestive cardiac failure, stroke and renal failure. The total cost to society was nearly $80 billion in 2010. The risk of death doubles for every 20 mm increase in systolic blood pressure above 120 mm. Conversely, a 5 mm reduction in systolic pressure reduces the risk of stroke by 14%, the risk of heart disease by 9% and the overall mortality by 7%.

Surgical sympathetic denervation for the treatment of resistant hypertension was routinely performed in the 1940's. Such procedures involved removing various combinations of ganglia in the neck, thoraco-lumbar spine, as well as excising the splanchnic nerve. Blood pressure decreases were very significant, and heart failure was improved. Such surgical procedures were also associated with significant procedural morbidity and mortality, however, and were rapidly abandoned in favor of pharmacologic treatments which became available in the 1950's. Pharmacotherapy became the mainstay of management for hypertensive patients during the second half of the last century. Many patients required more than one medication for adequate control of pressure and up to a quarter of all patients remained hypertensive on multiple medications (resistant hypertension).

Recently, mechanical means of controlling blood pressure have been revisited, specifically for patients with such resistant hypertension. Carotid sinus baroreceptor stimulation using implantable neurostimulation devices has been shown to reduce systolic pressures by up to 40 mm several years after the procedure. The only randomized clinical study using this device, however, missed the primary end-point and the study is being repeated. Furthermore, procedural complications attributable to the device were high. Renal artery denervation (RAD) is another new technique which involves ablating renal nerve fibres surrounding renal arteries bilaterally. The catheter is advanced into each of the renal arteries, and ablative energy is applied through the wall of the artery, to destroy some of the renal nerve fibres. The treatment lasts about 40 minutes. Procedure related complications are not uncommon. They include transient bradycardia, embolization from atheromatous renal arteries to kidneys whose function may already be impaired and renal artery spasm or dissection which may cause further deterioration in renal function. While both systolic and diastolic pressures improve following this treatment, the longer term effect on blood pressure is as yet unknown since peripheral nerve fibres such as those within the renal nerve typically regenerate. Such regeneration following radiofrequency ablation has been frequently demonstrated. After a significant portion of ablated fibres regenerate, the beneficial effect on blood pressure may be lost.

In this invention, we teach that denervation of cell bodies rather than nerve fibres is a more effective and long-lasting method of treating hypertension and related conditions, and furthermore may be easier to perform and safer than currently existing intra-arterial ablative techniques. Ganglion cells, unlike their axons in the renal nerve, don't regenerate. Thus blood pressure reduction is likely to be permanent. They are also tightly concentrated within the ganglia, such that ablating small portions of the ganglia can achieve greater blood pressure reduction than is possible by ablation within the renal artery. The pre-aortic ganglia are located on the antero-lateral aortic wall, cephalad and caudad to the superior mesenteric artery and closely adherent to the wall of the aorta. One method of denervating these cell bodies in accordance with the invention includes positioning a therapeutic ultrasound ablation device over the anterior abdominal wall, and using imaging techniques (CT, MRI, ultrasound) to adjust the beam depth such that it focuses on the pre-aortic ganglia and then ablating portions of these ganglia non-invasively. Another method of denervating pre-aortic ganglionic cell bodies involves laparoscopic insertion of an ablation device, advancing it ultrasonically to the pre-aortic ganglia, stimulating the ganglia and mechanically, chemically, electromagnetically, using say, radiofrequency or therapeutic ultrasound, ablating these structures. Yet another method would involve percutaneously advancing a needle through the anterior abdominal wall under imaging guidance and ablating the ganglia chemically, mechanically, electromagnetically or using therapeutic ultrasound. Yet another method would involve surgically opening the anterior abdominal wall and directly stimulating and ablating the pre-aortic ganglia or portions thereof, using any of the methods described above.

Those of skill in the art will appreciate that other similar modes of stimulation may be used and that the energy delivery device may be configured to stimulate or ablate tissue. Lastly, changes in arterial pressure may occur. After the ganglia are localized, the mode may be switched from electrical stimulation to focused ultrasound or to radiofrequency ablation and other modes known to those of skill in the art. Initially, this might cause BP to increase or decrease abruptly. The most significant advantages of this procedure over pharmacologic treatment alone or renal artery denervation (RAD) include significantly greater potential reductions in blood pressure and permanence of the hypotension achieved. The extent of the blood pressure reduction achieved is greater because the cell bodies whose axons are destined for the kidney are all very close together and the mechanism of action is different. Therefore ablation of even a small area in the relevant portion of the pre-aortic ganglia could destroy large numbers of cells whose axons are destined to the kidney and renal artery as well as other structures. Indeed dramatic drops in blood pressure and even postural hypotension have been reported following pharmacologic pre-aortic denervation in patients with intractable pain secondary to upper abdominal malignancies. In contrast, circumferential ablation of the renal nerve from within the renal artery destroys only a small fraction of the nerve fibres and only those destined to the kidney. Perhaps the most significant benefit of this technique is the permanent nature of the reduction in blood pressure. Destroyed ganglion cell bodies don't regenerate whereas destroyed nerve fibres do regenerate. Dead ganglion cell bodies disappear and are replaced in time by glial tissue. On the other hand, regeneration of nerve fibres following radiofrequency ablation has been documented. Significant regeneration could lead to the loss of the blood pressure reduction achieved early on following the procedure.

The inventors have found that these methods of treating hypertension are safer, simpler and less time-consuming than RAD. Instrumentation of the renal artery is often difficult, such that 15% of patients who would otherwise qualify for RAD cannot have it. Arterial stenosis, dissection, spasm and embolization to the kidneys of atheromatous material, all of which can cause deterioration in renal function, are not encountered during trans-abdominal pre-aortic ganglion cell ablation. Furthermore, complications associated with arterial access such as difficulty in instrumenting the femoral artery, long compression times, pseudoaneurysm formation and groin hematoma are all avoided using these methods. The pre-aortic ganglia are sizable structures and can be accurately imaged during the procedure, whether the ablation is done non-invasively or using percutaneous needle insertion or laparoscopy.

Although the present invention has been described with reference to preferred embodiments, workers skilled in the art will recognize that changes may be made in form and detail without departing from the spirit and scope of the invention.

We claim:

1. A method of modulating a physiological parameter of a patient, comprising percutaneously or transcutaneously disabling one or more pre-aortic ganglion cells within a pre-aortic ganglion via the anterior abdominal wall and improving said physiological parameter.

2. The method of claim 1 wherein said disabling comprises irreversibly disabling said one or more cells.

3. The method of claim 1 wherein improving said physiologic parameter comprises permanently improving said physiological parameter.

4. A method of modulating a physiological parameter of a patient, comprising destroying a pre-aortic ganglion cell to prevent regeneration.

5. The method of claim 1 wherein improving said physiological parameter comprises reducing blood pressure.

6. The method of claim 1 or 4 wherein the physiological parameter is associated with heart failure, hypertension, acute myocardial infarction, renal disease, chronic renal failure, obesity, diabetes, ischemic bowel syndrome, obstructive sleep apnea, disorders of intestinal motility, or peripheral vascular disease.

7. The method of claim 1 further comprising denervating only a portion of the pre-aortic ganglion including cells that innervate a kidney or an adrenal gland.

8. The method of claim 1 wherein disabling said one or more pre-aortic ganglion cells comprises applying an ablative electrical field to said pre-aortic ganglia.

9. The method of claim 1 further comprising stimulating said pre-aortic ganglion; monitoring a physiologic response related to said physiological parameter; applying an ablative energy to said one or more pre-aortic ganglion cells; and improving said physiological parameter.

10. The method of claim 9, wherein the physiologic response includes a change in blood pressure.

11. The method of claim 1 wherein said pre-aortic ganglion is selected from a celiac ganglion, mesenteric ganglion, suprarenal ganglion, inter-mesenteric ganglion, aortico-renal ganglion, and combinations of the foregoing.

12. The method of claim 1 further comprising providing an energy delivery device; positioning said energy delivery device over the anterior abdominal wall below the xiphisternum or percutaneously proximate the pre-aortic ganglion; and delivering energy.

13. The method of claim 12 further comprising imaging the pre-aortic ganglion during a procedure to modulate a physiological parameter of a patient.

14. The method of claim 14 wherein said imaging is external to the energy delivery device.

15. The method of claim 14 wherein said imaging comprises ultrasound delivered from said device.

16. The method of claim 9 further comprising stimulating the pre-aortic ganglion with an energy delivery device; and monitoring a blood pressure of the patient.

17. The method of claim 16 wherein monitoring said blood pressure includes monitoring a change in said blood pressure.

18. The method of claim 12 wherein delivering energy comprises delivering any wavelength from the electromagnetic spectrum, including radiofrequency, microwave, ultrasound, high intensity focused ultrasound, low intensity focused ultrasound, infrared waves, electrical energy, laser energy, other sources of thermal energy, and combinations of the foregoing.

19. The method of claim 18 wherein said thermal energy comprises cooling.

20. A method of modulating a physiological parameter of a patient comprising ablating a pre-aortic ganglia transcutaneously over an anterior abdominal wall.

21. The method of claim 20 wherein said ablating comprises using focused ultrasound.

22. The method of claim 21 wherein said focused ultrasound comprises high intensity focused ultrasound.

23. The method of claim 21 wherein said focused ultrasound comprises low intensity focused ultrasound.

24. A method of modulating a physiological parameter of a patient comprising ablating pre-aortic ganglia percutaneously through the anterior abdominal wall.

25. The method of claim 24 further comprising using a needle to perform said ablation.

26. The method of claim 25 wherein said needle delivers ultrasound to the pre-aortic ganglia.

27. The method of claim 25 wherein said needle deliver radiofrequency energy to the pre-aortic ganglia.

28. The method of claim 24 further comprising performing said ablation laparoscopically using a laparoscopic instrument including a camera.

29. The method of claim 24 further comprising using ultrasound to visualize said pre-aortic ganglia.

30. The method of claim 24 wherein said ablating is performed with a mechanical device.

31. The method of claim 24 wherein said ablating is performed using radiofrequency.

32. The method of claim 24 wherein said ablating is performed using ultrasound.

33. The method of claim 32 wherein said ablating is performed using a chemical agent.

34. The method of claim 33 wherein said chemical agent comprises phenol.
